(19) United States
(12) Patent Application Publication
(10) Pub. No.: US 2020/0251206 A1
(43) Pub. Date: Aug. 6, 2020

(54) SYSTEM AND METHOD FOR PLANNING A TIME SCHEDULE OF A MEDICAL EXAMINATION

(71) Applicant: Siemens Healthcare GmbH, Erlangen (DE)

(72) Inventor: Mario Zeller, Erlangen (DE)

(73) Assignee: Siemens Healthcare GmbH, Erlangen (DE)

(21) Appl. No.: 16/780,128

(22) Filed: Feb. 3, 2020

(30) Foreign Application Priority Data
Feb. 4, 2019 (DE) 102019201359.7

(51) Int. Cl. G16H 40/20; A61B 5/00; A61B 5/08; A61B 5/024; A61B 5/055; A61B 6/03; A61B 6/00; G16H 10/60; G16H 40/67; G16H 50/30

(52) U.S. Cl. CPC G16H 40/20; A61B 5/681; A61B 5/0816; A61B 5/02438; A61B 5/6898; A61B 5/055; A61B 2560/0431; A61B 6/037; A61B 6/5294; G16H 10/60; G16H 40/67; G16H 50/30; A61B 6/032

(57) ABSTRACT

Medical examinations, such as e.g. MRT recordings, are to be able to be better planned. To this end, in accordance with the disclosure, it is proposed that a physiological parameter of the patient be acquired before the medical examination. Subsequently there is an automated establishment of the time schedule of the medical examination as a function of the physiological parameter of the patient acquired.

### CROSS REFERENCE TO RELATED APPLICATIONS

This patent application claims priority to German Patent Application No. 102019201359.7, filed Feb. 4, 2019, which is incorporated herein by reference in its entirety.

### BACKGROUND

### Field

The present disclosure relates to a method for planning a time schedule of a medical examination of a patient with an examination facility. What is more the present disclosure relates to a system with an examination facility for medical examination of a patient. The present disclosure further relates to a corresponding computer program.

### Related Art

The success of imaging methods, of which the duration extends over several minutes, in particular of magnetic resonance examinations, is decisively dependent on physiological characteristics of the patient. As well as patient movement and the ability of the patient to hold their breath, depending on the desired examination, pulse and breathing frequency play a large role. There is therefore a plurality of what are known as triggered measurements, the sequence of which is dependent on breath curves or EKG curves acquired by sensors.

An example of a breath-triggered measurement is abdominal imaging with the TSE (Turbo Spin Echo) sequence or EPI (Echo Planar Imaging) diffusion sequence, for which the measurements are always to be recorded in the same breathing state, in order to exclude artifacts caused by breathing movement. Furthermore an EKG-triggered recording sequence of the heart can be obtained, in which a segmented motion recording of the complete heart cycle is to be made.

Depending on pulse or breathing frequency of the patient the sequence parameters have to be adapted or the recording strategy changed completely. Since pulse and breathing frequency of the patient are not known as a rule before the measurement, the strategy is only adapted in some cases when the measurements fail, which leads to an extended measurement time.

On the other hand a plurality of so-called “wearables” exists nowadays (e.g. fitness bracelets, Apple Watch, Samsung Gear), which collect a plurality of physiological data of the person wearing them. The minimum that is acquired is the pulse frequency over the entire day, or in some cases a simplified EKG curve is recorded. The current Apple Watch can detect heart rhythm problems for example. A few devices also acquire the breathing curve (e.g. a wearable device from Vandrico Inc.). Moreover possibilities exist for deducing the breathing rate from the pulse rate (cf. R. Bartsch et al., Phys Rev Lett 98, 054102, 2007. What is more, according to the German association for information technology, telecommunications and new media (bitkom), 30% of German citizens already have a fitness tracker in the form of a wearable.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 illustrates a flowchart of a method for planning examinations according to an exemplary embodiment of the present disclosure.

FIG. 2 illustrates a system for planning examinations according to an exemplary embodiment of the present disclosure.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure.

An object of the present disclosure can include improving the planning of the timing of a medical examination.

According to one aspect of the present disclosure, a method is thus provided for planning a time schedule of a medical examination of a patient with an examination facility. In an exemplary embodiment, the method includes acquiring a physiological parameter of the patient before the medical examination and establishing (e.g. an automated establishing) the time schedule of the medical examination as a function of the acquired physiological parameter of the patient.

The time schedule of a medical examination is thus to be planned. The time schedule can involve the start time, the end time or also a certain temporal structure of the medical examination. If in particular the medical examination is to be carried out in a timed series of individual steps, this timed series can accordingly be part of the planning.

The medical examination is done with an examination facility. The examination facility can comprise a complex examination device with corresponding electronic controller. In particular the examination facility can involve a facility for imaging, with which images can be obtained from inside a patient. The examination facility can however also involve non-imaging devices, with which simple breathing flows or airflows are measured for example.

In an exemplary embodiment, in a first step of the method includes acquiring a physiological parameter of the patient before the medical examination. Such a physiological parameter can involve a cyclic parameter, specifically the breathing frequency or the pulse frequency of a patient. The physiological parameter can however also be a multiple or a fraction of the actual physiological frequency. Thus the physiological parameter can be double the heart frequency or half the breathing frequency for example. The acquisition of this physiological parameter means for example that measured values of the parameter are obtained on the basis of time. For example the points in time are detected at which the patient has always breathed out completely. However the points in time can also be obtained for example at which the heart muscle is completed contracted. A significant aspect is that the physiological parameter of the patient has been acquired before the medical examination. In this way it can be ensured that the corresponding measured values are available for the planning of the time schedule of the medical examination, evaluated where necessary. If for example Magnetic Resonance Tomography (MRT) recordings of the patient are to be obtained, then the pulse of the patient should already be known before the MRT examination, so that the MRT examination can be planned or timed accordingly.

In an exemplary embodiment, in a further step of the method, the time schedule of the medical examination is established as a function of the physiological parameter of the patient acquired. In an exemplary embodiment, the established is automated. In an exemplary embodiment, the time schedule includes at least the overall duration of the medical examination. Thus, for a relative time of the beginning of the examination, a relative time of the end of the examination is also known. The entire sequence of the medical examination can also be composed of numerous individual steps. For example individual recordings of the patient are made at fixed intervals in time. If for example a pulse of 60 heartbeats per minute for the patient is established in this way and if 100 individual recordings are obtained, then the entire recording cycle lasts 1 minute and 40 seconds if one recording per heartbeat can be obtained. If necessary additional times for preparation and post-processing for the medical examination are to be included in the calculation. All in all this thus enables the timing of a triggered medical examination to be planned quite exactly.

In an exemplary embodiment, the physiological parameter of the patient is acquired with the aid of a portable device as an acquisition device. Such a portable device can be a professional device for acquiring a 24-hour EKG or a 24-hour blood pressure pulse measurement. However the portable device can also be a fitness pulse meter or another device that is primarily used by lay people. The data to be obtained about the physiological parameter can thus be obtained during the normal course of a patient's day in advance of the examination. A very convenient option is thus available for obtaining a dataset necessary for the planning of the examination.

In an exemplary embodiment, the portable device can involve a smartphone, a fitness bracelet, a wristwatch, a breathing frequency meter or a pulse meter. If necessary a smartphone on its own can record the necessary data or suitable smartphone accessories are needed to obtain this data. The data obtained can be buffered in an internal memory of the smartphone or the like and subsequently be transmitted to an evaluation unit with or without pre-processing. A fitness bracelet can record the pulse for example and buffer corresponding pulse data before it is read out via a corresponding interface. The same applies to a wristwatch. The breathing frequency meter can be a respiratory band for example, as is likewise usual in the fitness area. If necessary it is capable of recording and buffering both the breathing frequency and also the heart frequency. If necessary it should be ensured that the breathing frequency or the pulse frequency are measured in the resting state of the patient. Such a state would most preferably correspond to the examination situation when the patient is being examined by the examination facility. In heart imaging there are also so-called stress examinations, in which the heart rate is increased by a medicine. For this case the heart rate previously acquired under stress would also be helpful for the planning. In any event a suitable data interface should be provided on the portable device, in order to transfer the data about the physiological parameter obtained from the portable device to an external device and if necessary to the examination facility.

In an exemplary embodiment of the method, the medical examination comprises imaging steps. In the imaging the problem namely arises of the individual images as a rule representing states in different cycle states, so that the individual organs or sections of tissue assume different positions. So that the individual organs or sections of tissue always assume approximately the same position on the images, they are thus to be synchronized with the physiological cycle. As has already been shown above, this physiological cycle can be determined by the breathing period or the heart period or by their frequencies. Specifically with imaging methods an imaging step or an individual recording should thus be triggered with the respective physiological cycle. In this way for example a high-quality angiography can be carried out on the heart.

In an exemplary embodiment, the medical imaging examination includes a magnetic resonance tomography, a computed tomography or a positron emission tomography. Other imaging methods can also be planned and have their timing structured with the inventive method. In this way not only can the stress on the patient be minimized, but the imaging can also be operated more economically, since the overall obtaining of images can be carried out in the shortest time as a result of the stringent planning.

In an exemplary embodiment of the method, the physiological parameter is a cyclic parameter, such as a breathing frequency or a pulse frequency of the patient. In an exemplary embodiment, neither the breath nor the pulse can be held over a longer period as a rule. In an exemplary embodiment, the physiological parameter is alternatively a breathing period or a pulse period or fractions or multiples thereof. The physiological parameter can thus originate both from the frequency range and also from the time range. The suitable physiological parameter can be selected as a function of the data processing. As a rule the physiological parameter will always involve a repetitive movement however, as is given by the heartbeat or the breathing function.

What is more there can be provision in the inventive method that with the (automated) establishment of the time schedule of the medical examination an overall duration of the medical examination and/or a respective duration of part steps of the medical examination are obtained from the acquired physiological parameter. For example it can already be known for a specific medical examination that a specific number of imaging recordings is necessary, wherein a predetermined number of recordings per heartbeat or per number of heartbeats are possible. In this case only the individual number of heartbeats per minute of the patient would need to be multiplied by the facility-specific characteristic value of the examination facility, in order to establish the overall duration of the medical examination. In another example it can be necessary to determine the timing of an individual part step of the medical examination. This enables different medical examinations to be characterized with different numbers of part steps in respect of their respective overall duration. The time sequence of the medical method can thus be roughly identified by the overall duration or by the temporal structure on the basis of the time sections of the individual time steps of the medical examination.

In an exemplary embodiment of the inventive method, there can be provision for the acquisition of the physiological parameter of the patient to be done in a preliminary medical examination. Thus the physiological parameter does not have to be obtained by the patient themself, if necessary using means available to the layman, but can also be obtained in particular in a preliminary clinical examination by professional personnel and professional examination means, for example within the framework of a stress EKG. Specifically they can be provided in this way in a professional system, e.g. a hospital information system, for later examinations. What is more they can also be linked to other examination results of the patient. In particular it can be ensured in this way that they are also available for the planning of the impending examination.

In an exemplary embodiment of the method for planning a time schedule of a medical examination, the acquisition of the physiological parameter includes the fact that raw data is obtained in the portable device, this raw data is transferred wirelessly to an external device, is evaluated in the external device and is provided as evaluated data for the establishment of the time schedule of the medical examination. Consequently the portable device can involve a very simple device with which only the raw data is obtained. A complete evaluation does not usually take place in the portable device, but only in the external, i.e. independent, device (e.g. computing device). Minimal pre-processing of the raw data can still be undertaken in the portable device. The external device involves one or more servers for example, to which the data is transferred wirelessly or by wire. If the portable device is a smartphone, the obvious method of transmission is wireless. With simple measuring devices, such as for example fitness bracelets without a wireless interface, the data can be transmitted with corresponding cables to a PC or to another computing device, but also wirelessly to a smartphone. If necessary the data is forwarded from there to a server device in the Internet. The actual evaluation of the, if necessary, pre-processed, raw data is then done in the external device, i.e. the device which does not form a physical unit with the portable device. A pulse frequency or a breathing frequency is then obtained as the physiological parameter from said data for example. This parameter value is then provided for the planning of the examination. To this end the value provided can be requested or retrieved from planning software for example.

The medical raw data, which has been obtained by the portable device, typically involves personal, sensitive data. It is therefore of advantage for this raw data to be safeguarded by specific precautions. In particular there can be provision for the raw data only to be transmitted to the external device when the patient has granted this permission on the portable device. For example such a permission can be requested when the transmission of data to the external device is initiated. The permission can be given for example by a previously defined PIN. In general however the permission does not have to be given on the portable device. Instead it can be given on a preliminary examination device if the measured values for the physiological parameter are obtained within the framework of a preliminary examination.

In an exemplary embodiment, there can be provision for the acquisition of the physiological parameter to produce an irregularity in the timing of the physiological parameter, and therefore for the establishment of the time schedule of the medical examination not to be done as a function of the timing of the physiological parameter but of a predetermined value. If it is determined for example that the patient has very irregular breathing, the timing for examination steps (e.g. image recordings) cannot be established from this. In this case it is sensible under some circumstances not to plan the examination with fixed timing, but under some circumstances to perform other examination steps (e.g. propeller recordings) or other image processing means (e.g. averages).

In an exemplary embodiment of the method, the portable device additionally acquires a further parameter as well as the physiological parameter, which is included in the establishment of the time schedule of the medical examination. For example the portable device can have one or more further sensors, which provide further information about the patient. In this way for example a smartphone can have a position sensor with which additional movement data of the patient can be obtained during the acquisition of the physiological parameter. Such position data can give information for example about whether and the length of time for which a patient can hold still. Specifically the data can give information about whether the patient shakes or can hold still for a longer time. Furthermore it can be derived on the basis of the length of time or the relative rate of increase of the breathing or heart rate on transition from a rest situation to a stress situation whether a time buffer must be planned in when planning the examination, in order to avoid or having to repeat possible incorrect recordings as the nervousness of the patient increases during the medical examination.

According to the present disclosure a method can also be provided for carrying out a medical examination in accordance with a time schedule that has been planned as detailed above. The planned time schedule is thus employed directly in carrying out the medical examination. This means that this single medical examination on the one patient is structured and carried out according to the planned schedule. This enables the examination time to be minimized and the stress on the patient likewise to be optimized.

As has already been indicated above, the internal timing structure of the medical examination can likewise be made dependent on the acquired physiological parameter of the patient. This can then be implemented when the medical examination is carried out by the medical examination being timed at least in part as a function of a frequency of the acquired physiological parameter. Specifically the image recording frequency can be timed as a function of the heart frequency or the breathing frequency. In this way for example there can be a corresponding synchronization by an image always being recorded during a full contraction of the heart. But also for example in a heartbeat cycle a recording can be obtained in each case both with a full contraction and also during a complete relaxation of the heart muscle in each case. In this way the heartbeat cycle can also be subdivided multiple times if necessary. If obtaining the image or processing the image needs more time than the duration of a heartbeat, there can also be a synchronization or triggering by an image being recorded every two, three or four (etc.) heartbeats or being broken down into a number of shorter part recordings.

In an exemplary embodiment, a provision for one or more parameters of the examination facility is set as a function of the acquired physiological parameter. Thus if for example a specific pulse frequency is established as the physiological parameter for the patient, a parameter of the examination facility (e.g. exposure time, dose, number of recording events per pulse interval etc.) can be set as a function of the patient-specific, acquired physiological parameter. If necessary a number of different parameters of the examination facility are also set specifically for the patient in this way. Thus under some circumstances an entirely specific duration of the examination is produced for each individual patient.

In a development the physiological parameter can be monitored and/or recorded during the examination by the examination facility itself. In this way the physiological parameter obtained before the examination can be monitored if necessary for later evaluations during the examination. In this way for example the pulse obtained before the examination can be compared with a pulse during the examination. Consequences for later examinations can be established from this comparison. In this way it can be detected statistically how the examination is affecting the pulse beat of a patient, so that for a pulse estimation a pulse value obtained before the examination can be multiplied by a specific factor, since during the examination a correspondingly higher pulse will usually be measured. With a multiplication factor of this type the examination is able to be better planned in advance under some circumstances.

In an exemplary embodiment, a method for temporal planning of a series of examinations of a number of patients can likewise be provided, where the timing of at least one of the examinations is planned according to one of the methods presented above, and wherein a time for at least one further examination of the series of examinations is set as a function thereof. On the basis of the data obtained about their physiological parameter (e.g. heart frequency) before the examination of a patient, a conclusion can be drawn about the overall duration of the examination of this patient. Thus it can be decided for example whether the examination of this patient can be done in a time slot that is provided by two further examinations of other patients. In another example a number of examinations of different patients can be placed very close to each other in time, since their likely overall duration can be predicted very accurately with the aid of the inventive method. Specifically the beginning of a subsequent examination can be defined more accurately without any downtimes or maintenance times if the overall duration of a current examination is known in advance.

A system according to an exemplary embodiment includes:
an examination facility for medical examination of a patient and
an acquisition facility for acquiring a physiological parameter of the patient, wherein
the acquisition facility is independent of the examination facility and
the system has a processor for establishment of a time schedule of the medical examination as a function of the physiological parameter of the patient acquired.

The examination facility, as has already been mentioned above, can involve an imaging device for example, in particular an MRT device. The acquisition facility comprises a preliminary examination device for example, such as an EKG device for example, but also one of the devices mentioned above independent of the examination facility, such as a fitness bracelet, smartphone or the like. The processor for establishing the time schedule of the medical examination can be realized as a computer (e.g. personal computer) or processor circuitry that has a data connection to the acquisition facility, and, if necessary, also to the examination facility. In an exemplary embodiment, the processor includes storage means such as an internal and/or external memory. In an exemplary embodiment, data obtained from the patient can also be made available to the processor via a cloud (e.g. network, internet, etc.) for the planning.

The advantages and options for the aspects discussed above in conjunction with the inventive method can also be realized in an analogous way with the inventive system.

What is more a computer program is also provided in accordance with the disclosure, which can include commands that cause the above-mentioned system to carry out the method steps as have been explained above. One or more aspects of the present disclosure relate to a computer-readable medium, on which such a computer program is stored. The computer program can be used by one or more computers, processors or other systems executing commands. A computer-readable medium can be any device that contains, stores, communicates, transmits or transports the program for use by the respective device. The medium can involve an electronic, magnetic, optical, electromagnetic, infrared or semiconducting system. Examples of such media can be semiconductor or solid state memory, magnetic tape, computer diskettes, RAM memory, ROM memory, but also magnetic or optical disks. The processors and the program code can be implemented centrally or distributed.

In an exemplary embodiment, physiological patient data can be transmitted even before the (imaging) examination, using an interface, for example, to the examination facility (e.g. imaging modality) and to plan the examination in advance based on the physiological patient data.

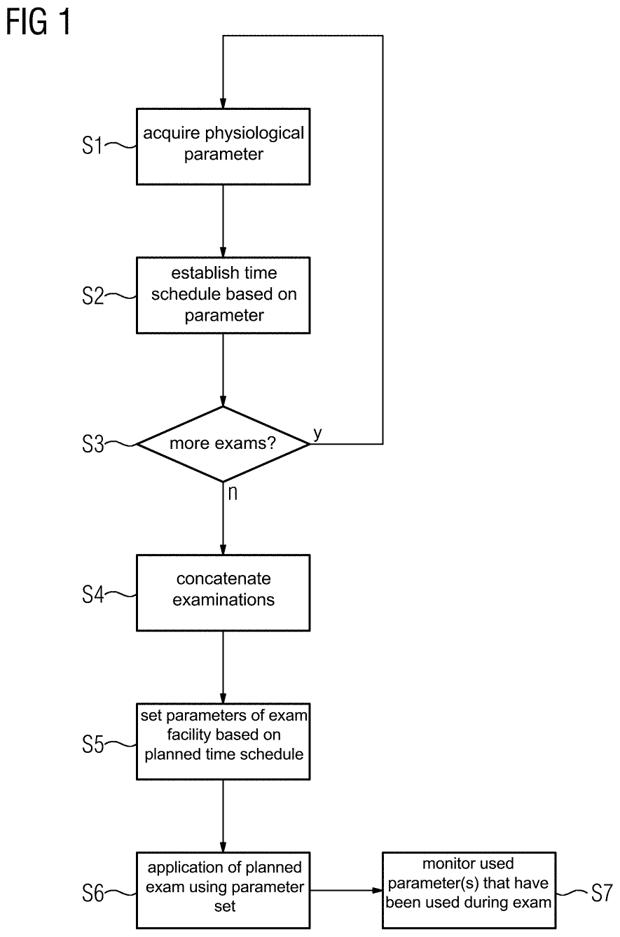

In the exemplary embodiment illustrated in FIG. 1, for the planning of a time schedule of a medical examination of a patient with an examination facility, initially in accordance with step S1 there is an acquisition of a physiological parameter of the patient before the medical examination. In particular, in this case the pulse frequency and/or the breathing frequency can be acquired as the physiological parameter with a portable device or “wearable,” that the patient is wearing. In a further step S2, the time schedule of the medical examination is established as a function of the acquired physiological parameter of the patient. This time schedule of the examination is preferably established in advance of this examination by means of a data processor that is different from the portable device. To this end, data is transferred where necessary from the portable device to the processor. Suitable wireless or wired interfaces are provided for this.

In the said way, an examination can be planned for a patient and the corresponding planning data can be provided at an output interface. However in the event of a number of examinations following on from one another in time and in particular when there are a number of patients to be examined, a check can be made in a step S3 as to whether corresponding further examinations are to take place. If this is the case (decision “y”), then the steps S1 and S2 are repeated correspondingly often for each examination or each patient. Thus the time schedule of each individual examination can be established.

In a step S4, after a negative decision “n” in step S3, all examinations are concatenated after one another in time. Since the time schedule of each individual examination and in particular its overall duration is known, the individual examinations can be concatenated to one another with a predetermined buffer for example. In an exemplary embodiment, a stringent schedule for a number of examinations one after the other is produced by this. In such cases the individual plans or individual sequences of the examinations are adapted individually for the respective patient. In an exemplary embodiment, in a subsequent optional step S5, parameters of the examination facility can be set according to the planned time schedules. For example the switching on and switching off of the x-ray tubes can be done according to the sequence established. Also for example the forwards movement of the table on which the patient is located can be controlled according to the time schedule.

In an exemplary embodiment, the actual application of the planned examination now takes place using the parameters set. This carrying out of the examination in accordance with a step S6 can be done by a unit other than the unit that has carried out the parameter acquisition or planning. While the planning has namely been carried out with a computing or evaluation device for example, an examination facility, such as an MRT device for example, can carry out the examination on the basis of the plan created. A permanent data connection between the examination facility and the processor creating the plan is not absolutely necessary to do this.

Thus the examination is carried out in step S6. In an exemplary embodiment, this involves an imaging method. In an exemplary embodiment, in an optional step S7, that parameter or those parameters of the patient that have been used during the examination for the planning of the examination are further monitored by a separate sensor system of the examination facility. In this way for example the heartbeat that serves as a basis for the planning of the examination can continue to be monitored during the examination. Statistics can be obtained from this additional data as to the effect that the examination itself is having on the patient for example.

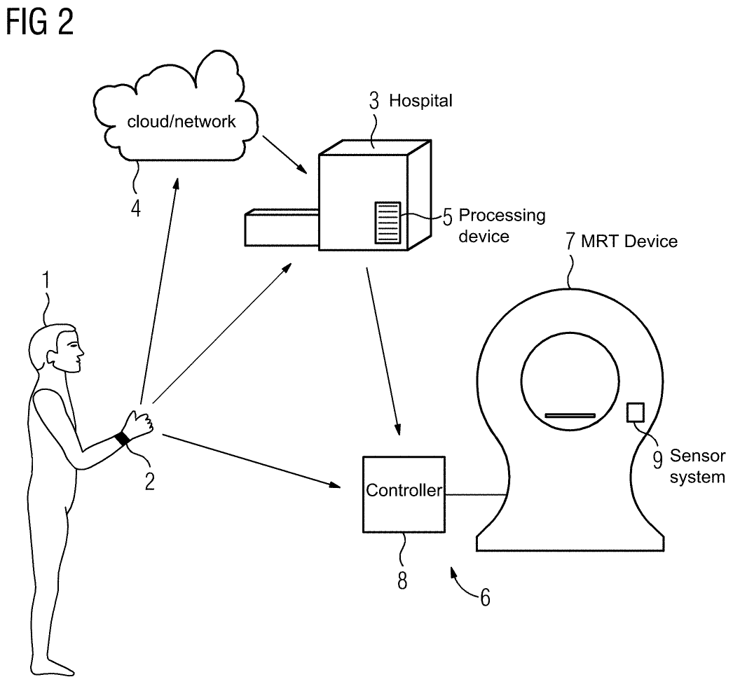

A system configured for examination planning according to an exemplary embodiment is shown in conjunction with FIG. 2. A patient 1 here is wearing a fitness bracelet 2 as the acquisition facility. With the fitness bracelet 2 the patient 1 records their heart frequency over a predetermined period of time. If necessary they must actuate the necessary facilities when doing so. The data recorded by the fitness bracelet 2 is transferred for example to a server or to a hospital 3. This data transmission can be done wirelessly or by wire. If necessary the data of the fitness bracelet 2 is also first read out in the hospital 3. The transmission of data to the server or to the hospital 3 can also be done via a cloud 4 for example. If necessary the data is buffered there if required.

In an exemplary embodiment, the hospital 3 includes a processing device 5. In an exemplary embodiment, the processing device 5 includes processor circuitry that is configured to perform one or more functions and/or operations of the processing device 5. In an exemplary embodiment, the processing device 5 is configured to plan an examination of the patient 1. In particular the data about the heart frequency of the patient 1 can be included for planning the timing of individual examination steps or also the entire examination sequence.

The patient is to be subjected to an MRT examination for example. This examination is to be done with the aid of an examination facility 6, which in the following example comprises an MRT device 7. The MRT device 7 is controlled by a controller 8 of the examination facility 6. This examination with the examination facility 6 can take place in the hospital 3 or at another location. The planning data of the processing device 5 are transferred to the controller 8 for this purpose. In an exemplary embodiment of the examination facility 6, there can also be provision for the controller 8 to be configured to directly record the data of the acquisition facility (here of the fitness bracelet 2) and process the data itself for planning of the examination. In this case, the planning of the examination thus takes place directly in the examination facility 6. In an exemplary embodiment, the controller 8 includes processor circuitry that is configured to perform one or more functions and/or operations of the controller 8.

If necessary, the examination facility 6 includes its own sensor system 9, with which one or more parameters of the patient 1 can be monitored during the examination. In an exemplary embodiment, this monitoring data involves the same data that has been used for the planning of the examination. For example the examination facility can have its own sensor system for detecting the heart frequency or the breathing frequency.

In an exemplary embodiment, data can originate on the one hand from devices that continuously record the data, such as wearables or smartphones, but on the other hand also from services that aggregate and prepare this data. The data for the examination planning can however also originate from preliminary medical examinations (e.g. pulse measurement or sleep laboratory measurement) and be made available for example via a hospital information system (HIS).

The physiological data recorded by wearables is evaluated as a rule on servers of the provider (e.g. FitBit or Apple) and can be displayed in an app. In this way, an authorized, selective enabling of the significant data directly by the patient would be possible (e.g. by means of authorized transmission of the service provider via a suitable communication technology and interrogation by the scanner), provided corresponding interfaces exist between the parties involved. In the very simplest form the user could also read their data directly from their device and the operators could enter it manually into the user interface of the scanner. A scanner is given here as an example of an MRT device, another imaging device or an examination facility in general.

In an exemplary embodiment, advantageously, the transmitted dataset includes a typical heart and breathing rate at rest and under stress, a current heart and breathing rate, information about the regularity of the breathing and also information about the regularity of the heartbeat. Standard deviations over different periods of time can be obtained for example to establish respective regularities. An especially precise automated adaptation of the scan parameters can be undertaken by means of this information.

For a breath-sensitive examination for example the current breathing rate serves as a preset for a suitable number of concatenations (i.e. the number of measurement steps into which a multislice recording must be divided, in order to be able to take place in each case in a similar breathing state). For example if a slice stack with 20 to 30 slices is to be recorded, wherein one second is needed for each slice. If the patient for example, in accordance with data obtained in advance, has a breathing cycle duration of five seconds, there is the possibility of recording a maximum of five slices during a breathing cycle. In a subsequent new breathing cycle a further five slices can be recorded until ultimately all slices are recorded.

In another exemplary embodiment, the recordings are triggered according to the heart rate. This breathing rate is obtained individually from the patient and amounts to four seconds for example. If necessary two recordings can be obtained per breathing cycle, namely during full breathing-in (inspiration) and during full breathing-out (expiration). Thus a total of two recordings can be made in four seconds. The overall examination can be planned accordingly.

Typical breathing rates at rest and under stress make it possible to plan in a buffer if the patient becomes more nervous as a result of the restricted environment, e.g. in the MR scanner and thus accelerates their breathing frequency. Information about the regularity of the breathing allows an assessment of whether, instead of a breath triggering, another strategy for the examination is to be selected. If for example the breathing is very irregular, then the examination can be planned such that each slice is measured a number of times when the patient is breathing freely and appropriate averagings are performed. With entirely irregular breathing however what are known as propeller recordings can also be carried out, wherein numerous radial recordings are obtained perpendicular to the plane of the slice. Similar considerations apply for planning with heart rate information.

Since wearables have a plurality of sensors, in particular position sensors, the raw data of these sensors can provide additional decisive information for the scan planning, e.g. whether a patient can hold their arm still for a long period of time or continually shakes. In the first case fast breath-holding sequences for example could be selected during examination planning; in the latter case movement-sensitive imaging sequences could be selected.

In an exemplary embodiment, by comparing the values delivered by wearables with the breathing and heart rates acquired by sensors of the examination facility or of the scanner during the examination, scan planning can be further improved for the future. In this way for example it can be checked whether the data of the wearable matches the data of the sensors at the examination facility. In particular for example a more precise age, weight or gender-specific prediction about a possible deviation of the heart rate actually achieved because of nervousness during the examination can be made. The examples given above occur specifically for an MR examination. The method can however also be employed for other examinations and specifically for other imaging modalities, which are sensitive to physiological influences, e.g. CT or PET.

In an advantageous manner, with the aid of the present disclosure, physiological data recorded by wearables can now be used by means of an interface for examination planning. Thus examinations better tailored to the respective patient and thus a higher image quality and a reduction in the likelihood of repeated measurements are possible. What is more, however an enhanced convenience for the patient is produced by “dividing up” their health information. However, since sensitive information is involved, an appropriate action of the patient should be provided to enable the data needed to be used. In particular however the disclosure also allows the examination facilities to be better utilized, since a more precise planning of the individual examinations is possible.

Where this has not yet been explicitly done, but is sensible and in the spirit of the disclosure however, individual exemplary embodiments, individual of their part aspects or features can be combined with one another or exchanged, without departing from the framework of the disclosure given here. Advantages of the disclosure described with regard to one exemplary embodiment, where they are able to be transferred, also apply, without being explicitly named, to other exemplary embodiments.

Any connection or coupling between functional blocks, devices, components of physical or functional units shown in the drawings and described hereinafter may be implemented by an indirect connection or coupling. A coupling between components may be established over a wired or wireless connection. Functional blocks may be implemented in hardware, software, firmware, or a combination thereof.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general purpose computer.

For the purposes of this discussion, the term “processor circuitry” shall be understood to be circuit(s), processor(s), logic, or a combination thereof. A circuit includes an analog circuit, a digital circuit, state machine logic, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A method for planning a time schedule of a medical examination of a patient with an examination facility, the method comprising:
acquiring a physiological parameter of the patient before the medical examination; and
automatically establishing the time schedule of the medical examination as a function of the acquired physiological parameter of the patient.

2. The method as claimed in claim 1, wherein the acquisition of the physiological parameter of the patient is performed using a portable device.

3. The method as claimed in claim 2, wherein the portable device is a smartphone, a fitness bracelet, a wristwatch, a breathing frequency meter, or a pulse meter.

4. The method as claimed in claim 1, wherein the medical examination comprises one or more to be planned imaging operations of the patient.

5. The method as claimed in claim 4, wherein the medical examination comprises a magnetic resonance tomography, a computed tomography, or a positron emission tomography.

6. The method as claimed in claim 1, wherein the physiological parameter is a cyclic parameter.

7. The method as claimed in claim 6, wherein the cyclic parameter comprises a breathing frequency or a pulse frequency of the patient.

8. The method as claimed in claim 1, wherein, when the time schedule of the medical examination is established, an overall duration of the medical examination and/or a respective duration of a portion of the medical examination is obtained from the acquired physiological parameter.

9. The method as claimed in claim 1, wherein the acquisition of the physiological parameter of the patient comprises acquiring the physiological parameter of the patient in a preliminary medical examination.

10. The method as claimed in claim 2, wherein the acquisition of the physiological parameter includes acquiring raw data in the portable device, the acquired raw data being transferred to an external device, evaluated in the external device, and provided as evaluated data, wherein the establishment of the time schedule of the medical examination being based on the evaluated data.

11. The method as claimed in claim 9, wherein the raw data is only transmitted to the external device in response to the patient granting permission on the portable device for transmission of the raw data.

12. The method as claimed in claim 1, wherein, in the acquisition of the physiological parameter, in response to an irregularity in the timing of the physiological parameter, the time schedule of the medical examination is established as a function of a predetermined value instead of as a function of the timing of the physiological parameter.

13. The method as claimed in claim 2, wherein the portable device additionally acquires a further parameter that is included in the establishment of the time schedule of the medical examination.

14. A method for carrying out a medical examination according to a time schedule, which is planned in accordance with the method as claimed in claim 1.

15. The method as claimed in claim 14, wherein the medical examination is at least partly timed by a frequency of the physiological parameter acquired.

16. The method as claimed in claim 14, wherein one or more parameters of the examination facility are set as a function of the physiological parameter acquired.

17. The method as claimed in claim 14, wherein the physiological parameter is monitored and/or recorded during the examination by the examination facility.

18. A method for planning the timing of a series of examinations of a number of patients, wherein the timing of at least one of the examinations is planned according to a method as claimed in claim 1, wherein at least one further examination of the series of examinations is scheduled as a function thereof.

19. A non-transitory computer-readable storage medium with an executable program stored thereon, that when executed, instructs a processor to perform the method of claim 1.

20. A computer program product having a computer program which is directly loadable into a memory of a controller of the magnetic resonance imaging system, when executed by the controller, causes the magnetic resonance system to perform the method as claimed in claim 1.

21. A system for planning a time schedule of a medical examination, comprising:
an examination facility for medical examination of a patient;
an acquisition facility independent of the examination facility and configured to acquire a physiological parameter of the patient; and
a processor configured to establish the time schedule of the medical examination as a function of the acquired physiological parameter of the patient.
